(19) United States
(12) Patent Application Publication
(10) Pub. No.: US 2009/0005435 A1
(43) Pub. Date: Jan. 1, 2009

(54) Novel Compounds, Pharmaceutical Compositions Containing Same, and Methods of Use for Same

(76) Inventors: Jill M. Sturdivant, Baltimore, MD (US); Craig Townsend, Baltimore, MD (US); Susan Medghalchi, Baltimore, MD (US)

Correspondence Address: JACOBSON HOLMAN PLLC, 400 SEVENTH STREET N.W., SUITE 600, WASHINGTON, DC, 20004, US

(21) Appl. No.: 11/597,538

(22) PCT Filed: May 25, 2005
(86) PCT No.: PCT/US05/18443

§ 371 Date: Apr. 29, 2008

Related U.S. Application Data
(60) Provisional application No. 60/574,639, filed on May 26, 2004.

(51) Int. Cl. A61K 31/381; C07D 333/32; A61P 35/00; A61P 3/04

(52) U.S. Cl. 514/445; 549/62

(57) ABSTRACT

A pharmaceutical composition comprising a pharmaceutical diluent and a compound of formula (II), wherein R¹ and R², the same or different from each other, are H, C₁-C₂₀ alkyl, cycloalkyl, alkenyl, aryl, arylalkyl, or alkylaryl, —CH₂COR^{S}, —CH₂C(O)NR^{S}, —C(O)R⁵, or —CH₂OR⁵, and can optionally contain halogen atoms, where R⁵ is a C₁-C₁₂ alkyl group. R³ and R⁴, the same or different from each other, are H, C₁-C₂₀ alkyl cycloalkyl, alkenyl, aryl, arylalkyl, or alkylaryl.

### BACKGROUND OF THE INVENTION

### Fatty Acid Synthase

Fatty acids have three primary roles in the physiology of cells. First, they are the building bocks of biological membranes. Second, fatty acid derivatives serve as hormones and intracellular messengers. Third, and of particular importance to the present invention, fatty acids are fuel molecules that can be stored in adipose tissue as triacylglycerols, which are also known as neutral fats.

There are four primary enzymes involved in the fatty acid synthetic pathway, fatty acid synthase (FAS), acetyl-CoA carboxylase (ACC), malic enzyme, and citric lyase. The principal enzyme, FAS, catalyzes the NADPH-dependent condensation of the precursors malonyl-CoA and acetyl-CoA to produce fatty acids. NADPH is a reducing agent that generally serves as the essential electron donor at two points in the reaction cycle of FAS. The other three enzymes (i.e., ACC, malic enzyme, and citric lyase) produce the necessary precursors. Other enzymes, for example the enzymes that produce NADPH, are also involved in fatty acid synthesis.

FAS has an Enzyme Commission (E.C.) No. 2.3.1.85 and is also known as fatty acid synthetase, fatty acid ligase, as well as its systematic name acyl-CoA:malonyl-CoA C-acyltransferase (decarboxylating, oxoacyl- and enoyl-reducing and thioester-hydrolysing). There are seven distinct enzymes—or catalytic domains—involved in the FAS catalyzed synthesis of fatty acids: acetyl transacylase, malonyl transacylase, beta-ketoacyl synthetase (condensing enzyme), beta-ketoacyl reductase, beta-hydroxyacyl dehydrase, enoyl reductase, and thioesterase. (Wakil, S. J., Biochemistry, 28: 4523-4530, 1989). All seven of these enzymes together form FAS.

Although the FAS catalyzed synthesis of fatty acids is similar in lower organisms, such as, for example, bacteria, and in higher organisms, such as, for example, mycobacteria, yeast and humans, there are some important differences. In bacteria, the seven enzymatic reactions are carried out by seven separate polypeptides that are non-associated. This is classified as Type II FAS. In contrast, the enzymatic reactions in mycobacteria, yeast and humans are carried out by multifunctional polypeptides. For example, yeast have a complex composed of two separate polypeptides whereas in mycobacterium and humans, all seven reactions are carried out by a single polypeptide. These are classified as Type I FAS.

### FAS Inhibitors

Various compounds have been shown to inhibit fatty acid synthase (FAS). FAS inhibitors can be identified by the ability of a compound to inhibit the enzymatic activity of purified FAS. FAS activity can be assayed by measuring the incorporation of radiolabeled precursor (i.e., acetyl-CoA or malonyl-CoA) into fatty acids or by spectrophotometrically measuring the oxidation of NADPH. (Dils, et al., Methods Enzymol., 35:74-83).

Table 1, set forth below, lists several FAS inhibitors.

TABLE 1
Representative Inhibitors Of The Enzymes Of The Fatty Acid Synthesis
Pathway

Inhibitors of Fatty Acid Synthase
1,3-dibromopropanone
Ellman's reagent (5,5′-dithiobis(2-nitrobenzoic acid), DTNB)
4-(4′-chlorobenzyloxy) benzyl nicotinate (KCD-232)
4-(4′-chlorobenzyloxy) benzoic acid (MII)
2(5(4-chlorophenyl)pentyl)oxirane-2-carboxylate (POCA) and its CoA
derivative
ethoxyformic anhydride
cerulenin
phenyocerulenin
melarsoprol
iodoacetate
phenylarsineoxide
pentostam
melittin
thiolactomycin
Inhibitors for citrate lyase
(−) hydroxycitrate
(R,S)-S-(3,4-dicarboxy-3-hydroxy-3-methyl-butyl)-CoA
S-carboxymethyl-CoA
Inhibitors for malic enzyme
periodate-oxidized 3-aminopyridine adenine dinucleotide phosphate
5,5′-dithiobis(2-nitrobenzoic acid)
p-hydroxymercuribenzoate
N-ethylmaleimide
oxalyl thiol esters such as S-oxalylglutathione
gossypol
phenylglyoxal
2,3-butanedione
bromopyruvate
pregnenolone
Inhibitors for acetyl CoA carboxylase
sethoxydim
haloxyfop and its CoA ester
diclofop and its CoA ester
clethodim
alloxydim
trifop
clofibric acid
2,4-D mecoprop
dalapon
2-alkyl glutarate
2-tetradecanylglutarate (TDG)
2-octylglutaric acid
N6,02-dibutyryl adenosine cyclic 3′,5′-monophosphate
N2,02-dibutyryl guanosine cyclic 3′,5′-monophosphate
CoA derivative of 5-(tetradecyloxy)-2-furoic acid (TOFA)
2,3,7,8-tetrachlorodibenzo-p-dioxin
9-decenyl-1-pentenedioic acid
decanyl-2-pentenedioic acid
decanyl-1-pentenedioic acid
(S)-ibuprofenyl-CoA
(R)-ibuprofenyl-CoA
fluazifop and its CoA ester
clofop
5-(tetradecycloxy)-2-furoic acid
beta, beta′-tetramethylhexadecanedioic acid
tralkoxydim
free or monothioester of beta, beta prime-methyl-
substituted hexadecanedioic acid (MEDICA 16)
alpha-cyanco-4-hydroxycinnamate
S-(4-bromo-2,3-dioxobutyl)-CoA
p-hydroxymercuribenzoate (PHMB)
N6,02-dibutyryl adenosine cyclic 3′,5′-monophosphate

Of the four enzymes in the fatty acid synthetic pathway, FAS is the preferred target for inhibition because it acts only within the pathway to fatty acids, while the other three enzymes are implicated in other cellular functions. Therefore, inhibition of one of the other three enzymes is more likely to affect normal cells. Of the seven enzymatic steps carried out by FAS, the step catalyzed by the condensing enzyme (i.e., beta-ketoacyl synthetase) and the enoyl reductase have been the most common candidates for inhibitors that reduce or stop fatty acid synthesis. The condensing enzyme of the FAS complex is well characterized in terms of structure and function. The active site of the condensing enzyme contains a critical cysteine thiol, which is the target of antilipidemic reagents, such as, for example, the inhibitor cerulenin.

Preferred inhibitors of the condensing enzyme include a wide range of chemical compounds, including alkylating agents, oxidants, and reagents capable of undergoing disulphide exchange. The binding pocket of the enzyme prefers long chain, E, E, dienes.

In principal, a reagent containing the sidechain diene and a group which exhibits reactivity with thiolate anions could be a good inhibitor of the condensing enzyme. Cerulenin [(2S,3R)-2,3-epoxy-4-oxo-7,10 dodecadienoyl amide] is an example:

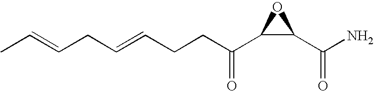

Cerulenin covalently binds to the critical cysteine thiol group in the active site of the condensing enzyme of fatty acid synthase, inactivating this key enzymatic step (Funabashi, et al., J. Biochem., 105:751-755, 1989). While cerulenin has been noted to possess other activities, these either occur in microorganisms which may not be relevant models of human cells (e.g., inhibition of cholesterol synthesis in fungi, Omura (1976), Bacteriol. Rev., 40:681-697; or diminished RNA synthesis in viruses, Perez, et al. (1991), FEBS, 280: 129-133), occur at a substantially higher drug concentrations (inhibition of viral HIV protease at 5 mg/ml, Moelling, et al. (1990), FEBS, 261:373-377) or may be the direct result of the inhibition of endogenous fatty acid synthesis (inhibition of antigen processing in B lymphocytes and macrophages, Falo, et al. (1987), J. Immunol., 139:3918-3923). Some data suggest that cerulenin does not specifically inhibit myristoylation of proteins (Simon, et al., J. Biol. Chem., 267:3922-3931, 1992).

Several more FAS inhibitors are disclosed in U.S. patent application Ser. No. 08/096,908 and its CIP filed Jan. 24, 1994, the disclosures of which are hereby incorporated by reference. Included are inhibitors of fatty acid synthase, citrate lyase, CoA carboxylase, and malic enzyme.

Tomoda and colleagues (Tomoda et. al., Biochim. Biophys. Act 921:595-598 1987; Omura el. al., J. Antibiotics 39:1211-1218 1986) describe Triacsin C (sometimes termed WS-1228A), a naturally occurring acyl-CoA synthetase inhibitor, which is a product of Streptomyces sp. SK-1894. The chemical structure of Triacsin C is 1-hydroxy-3-(E,E,E-2′,4′,7′-undecatrienylidine) triazene. Triacsin C causes 50% inhibition of rat liver acyl-CoA synthetase at 8.7 μM; a related compound, Triacsin A, inhibits acyl CoA-synthetase by a mechanism which is competitive with long-chain fatty acids. Inhibition of acyl-CoA synthetase is toxic to animal cells. Tomoda et al. (Tomoda el. al., J. Biol. Chem. 266:4214-4219, 1991) teaches that Triacsin C causes growth inhibition in Raji cells at 1.0 μM, and have also been shown to inhibit growth of Vero and Hela cells. Tomoda el. al. further teaches that acyl-CoA synthetase is essential in animal cells and that inhibition of the enzyme has lethal effects.

A family of compounds (gamma-substituted-alpha-methylene-beta-carboxy-gamma-butyrolactones) has been shown in U.S. Pat. No. 5,981,575 (the disclosure of which is hereby incorporated by reference) to inhibit fatty acid synthesis, inhibit growth of tumor cells, and induce weight loss. The compounds disclosed in the '575 patent have several advantages over the natural product cerulenin for therapeutic applications: [1] they do not contain the highly reactive epoxide group of cerulenin, [2] they are stable and soluble in aqueous solution, [3] they can be produced by a two-step synthetic reaction and thus easily produced in large quantities, and [4] they are easily tritiated to high specific activity for biochemical and pharmacological analyses. The synthesis of this family of compounds, many of which are fatty acid synthase inhibitors, is described in the '575 patent, as is their use as a means to treat tumor cells expressing FAS, and their use as a means to reduce body weight. The '575 patent also discloses the use of any fatty acid synthase inhibitors to systematically reduce adipocyte mass (adipocyte cell number or size) as a means to reduce body weight.

Other disclosures of FAS-inhibiting compounds include patent applications PCT/US03/20960 and PCT/US03/21700, the disclosures of which are hereby incorporated by reference.

The primary sites for fatty acid synthesis in mice and humans are the liver (see Roncari, Can. J. Biochem., 52:221-230, 1974; Triscari et al., 1985, Metabolism, 34:580-7; Barakat et al., 1991, Metabolism, 40:280-5), lactating mammary glands (see Thompson, et al., Pediatr. Res., 19:139-143, 1985) and adipose tissue (Goldrick et al., 1974, Clin. Sci. Mol. Med., 46:469-79).

### Inhibitors of Fatty Acid Synthesis as Antimicrobial Agents

Cerulenin was originally isolated as a potential antifungal antibiotic from the culture broth of Cephalosporium caerulens. Structurally cerulenin has been characterized as (2R,3S)-epoxy-4-oxo-7,10-trans,trans-dodecanoic acid amide. Its mechanism of action has been shown to be inhibition, through irreversible binding, of beta-ketoacyl-ACP synthase, the condensing enzyme required for the biosynthesis of fatty acids. Cerulenin has been categorized as an antifungal, primarily against Candida and Saccharomyces sp. In addition, some in vitro activity has been shown against some bacteria, actinomycetes, and mycobacteria, although no activity was found against Mycobacterium tuberculosis. The activity of fatty acid synthesis inhibitors and cerulenin in particular has not been evaluated against protozoa such as Toxoplasma gondii or other infectious eucaryotic pathogens such as Pneumocystis carinii, Giardia lamblia, Plasmodium sp., Trichomonas vaginalis, Cryptosporidium, Trypanosoma, Leishmania, and Schistosoma.

Infectious diseases which are particularly susceptible to treatment are diseases which cause lesions in externally accessible surfaces of the infected animal. Externally accessible surfaces include all surfaces that may be reached by non-invasive means (without cutting or puncturing the skin), including the skin surface itself, mucus membranes, such as those covering nasal, oral, gastrointestinal, or urogenital surfaces, and pulmonary surfaces, such as the alveolar sacs. Susceptible diseases include: (1) cutaneous mycoses or tineas, especially if caused by Microsporum, Trichophyton, Epidermophyton, or Mucocutaneous candidiasis; (2) mucotic keratitis, especially if caused by Aspergillus, Fusarium or Candida; (3) amoebic keratitis, especially if caused by Acanthamoeba; (4) gastrointestinal disease, especially if caused by Giardia lamblia, Entamoeba, Cryptosporidium, Microsporidium, or Candida (most commonly in immunocompromised animals); (5) urogenital infection, especially if caused by Candida albicans or Trichomonas vaginalis; and (6) pulmonary disease, especially if caused by Mycobacterium tuberculosis, Aspergillus, or Pneumocystis carinii. Infectious organisms that are susceptible to treatment with fatty acid synthesis inhibitors include Mycobacterium tuberculosis, especially multiply-drug resistant strains, and protozoa such as Toxoplasma.

Any compound that inhibits fatty acid synthesis may be used to inhibit microbial cell growth. However, compounds administered to a patient must not be equally toxic to both patient and the target microbial cells. Accordingly, it is beneficial to select inhibitors that only, or predominantly, affect target microbial cells.

Eukaryotic microbial cells which are dependent on their own endogenously synthesized fatty acid will express Type I FAS. This is shown both by the fact that FAS inhibitors are growth inhibitory and by the fact that exogenously added fatty acids can protect normal patient cells but not these microbial cells from FAS inhibitors. Therefore, agents which prevent synthesis of fatty acids by the cell may be used to treat infections. In eukaryotes, fatty acids are synthesized by Type I FAS using the substrates acetyl CoA, malonyl CoA and NADPH. Thus, other enzymes which can feed substrates into this pathway may also effect the rate of fatty acid synthesis and thus be important in microbes that depend on endogenously synthesized fatty acid. Inhibition of the expression or activity of any of these enzymes will effect growth of the microbial cells that are dependent upon endogenously synthesized fatty acid.

The product of Type I FAS differs in various organisms. For example, in the fungus S. cerevisiae the products are predominately palmitate and sterate sterified to coenzyme-A. In Mycobacterium smegmatis, the products are saturated fatty acid CoA esters ranging in length from 16 to 24 carbons. These lipids are often further processed to fulfill the cells need for various lipid components.

Inhibition of key steps in down-stream processing or utilization of fatty acids may be expected to inhibit cell function, whether the cell depends on endogenous fatty acid or utilizes fatty acid supplied from outside the cell, and so inhibitors of these down-stream steps may not be sufficiently selective for microbial cells that depend on endogenous fatty acid. However, it has been discovered that administration of Type I fatty acid synthesis inhibitor to such microbes makes them more sensitive to inhibition by inhibitors of down-stream fatty acid processing and/or utilization. Because of this synergy, administration of a fatty acid synthesis inhibitor in combination with one or more inhibitors of down-stream steps in lipid biosynthesis and/or utilization will selectively affect microbial cells that depend on endogenously synthesized fatty acid. Preferred combinations include an inhibitor of FAS and acetyl CoA carboxylase, or FAS and an inhibitor of MAS.

When it has been determined that a mammal is infected with cells of an organism which expresses Type I FAS, or if FAS has been found in a biological fluid from a patient, the mammal or patient may be treated by administering a fatty acid synthesis inhibitor (U.S. Pat. No. 5,614,551).

The inhibition of neuropeptide-Y to depress appetite and stimulate weight loss is described in International Patent Application No. PCT/US01/05316 the disclosure of which is hereby incorporated by reference. That application, however, does not describe or disclose any of the compounds disclosed in the present application

The stimulation of carnitine palmitoyl transferase-1 (CPT-1) to stimulate weight loss is described in U.S. Patent Application Ser. No. 60/354,480, the disclosure of which is hereby incorporated by reference. That application does not describe or disclose any of the compounds disclosed herein, either.

The use of FAS inhibitors to inhibit the growth of cancer cells is described in U.S. Pat. No. 5,759,837, the disclosure of which is hereby incorporated by reference. That application does not describe or disclose any of the compounds disclosed herein.

### SUMMARY OF THE INVENTION

It is an object of this invention to provide a new class of compounds of formula I which have a variety of therapeutically valuable properties, eg. FAS-inhibition, NPY-inhibition, CPT-1 stimulation, ability to induce weight loss, and anti-cancer and anti-microbial properties.

It is a further object of this invention to provide pharmaceutical compositions comprising compounds of formula II and a pharmaceutical diluent.

It is a further object of this invention to provide a method of inducing weight loss in animals and humans by administering a pharmaceutical composition comprising compounds of formula II and a pharmaceutical diluent.

It is a further object of the invention to provide a method of stimulating the activity of CPT-1 by administering to humans or animals pharmaceutical composition comprising compounds of formula II and a pharmaceutical diluent.

It is a further object of the invention to provide a method of inhibiting the synthesis of neuropeptide Y in humans or animals by administering a pharmaceutical composition comprising compounds of formula II and a pharmaceutical diluent.

It is a further object of the invention to provide a method of inhibiting fatty acid synthase activity in humans or animals by administering pharmaceutical composition comprising compounds of formula II and a pharmaceutical diluent.

It is a further object of this invention to provide a method of treating cancer in animals and humans by administering a pharmaceutical composition comprising compounds of formula II and a pharmaceutical diluent.

It is still a further object of this invention to provide a method of preventing the growth of cancer cells in animals and humans by administering a pharmaceutical composition comprising compounds of formula II and a pharmaceutical diluent.

It is a further object of this invention to provide a method of inhibiting growth of invasive microbial cells by administering pharmaceutical composition comprising compounds of formula II and a pharmaceutical diluent.

### BRIEF DESCRIPTION OF THE DRAWINGS

FIG. 1 shows a synthetic scheme to make a compound according to the invention.

FIG. 2 shows another synthetic scheme to make compounds according to the invention.

### DETAILED DESCRIPTION OF THE INVENTION

The compounds of the invention can be prepared by conventional means. The synthesis of a number of the compounds is described in the examples. The compounds may be useful for the treatment of obesity, cancer, or microbially-based infections.

One embodiment of the invention is compounds having the following general formula:

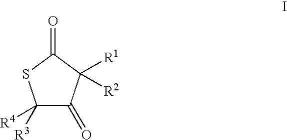

wherein:
R¹ and R², the same or different from each other, are H, C₁-C₂₀ alkyl, cycloalkyl, alkenyl, aryl, arylalkyl, or alkylaryl, —CH₂COR⁵, —CH₂C(O)NR⁵, —C(O)R⁵, or —CH₂OR⁵, and can optionally contain halogen atoms, where R⁵ is a C₁-C₁₂ alkyl group;
R³ and R⁴, the same or different from each other, are H, C₁-C₂₀ alkyl, cycloalkyl, alkenyl, aryl, arylalkyl, or alkylaryl;
with the proviso that when R⁴═—(CH₂)₇CH₃, R³ is methyl, and R¹ is —CH₃, R² is not —CH₂—CH═CH₂,
and with the further proviso that when R⁴═—CH₃, R³ is H, and R¹ is —CH₃, R² is not —CH₃, or —CH═C(CH₃)CH₂CH₂CH═C(CH₃)₂.

Preferably, R¹ and R² are each independently C₁-C₁₂ alkyl. In a preferred embodiment, R¹ and R² are each —CH₂—CH═CH₂.

Preferably, R³ and R⁴ are each independently a C₁-C₁₂ alkyl group. More, preferably, R⁴ is a C₁-C₈ alkyl group, most preferably —CH₃.

The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

The clinical therapeutic indications envisioned for the compounds of the invention include: (1) infections due to invasive micro-organisms such as staphylococci and enterococci; (2) cancers arising in many tissues whose cells over-express fatty acid synthase, and (3) obesity due to the ingestion of excess calories. Dose and duration of therapy will depend on a variety of factors, including (1) the patient's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, doses will be chosen to achieve serum levels of 1 ng/ml to 100 ng/ml with the goal of attaining effective concentrations at the target site of approximately 1 μg/ml to 10 μg/ml.

### EXAMPLES

The invention will be illustrated, but not limited, by the following examples:

A compound according to the invention were synthesized as described below. Biological activity of the compound was profiled as follows: It was tested for: (1) inhibition of purified human FAS, (2) inhibition of fatty acid synthesis activity in whole cells and (3) cytotoxicity against cultured MCF-7 human breast cancer cells, known to possess high levels of FAS and fatty acid synthesis activity, using the crystal violet and XTT assays. Select compounds with low levels of cytotoxicity were then tested for weight loss in Balb/C mice. In addition, a representative compound from the group which exhibited significant weight loss and low levels of cytotoxicity was tested for its effect on fatty acid oxidation, and carnitine palmitoyltransferase-1 (CPT-1) activity, as well as hypothalamic NPY expression by Northern analysis in Balb/C mice. Certain compounds were also tested for activity against gram positive and/or negative bacteria.

### Chemical Synthesis of Compounds

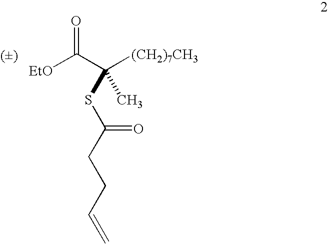

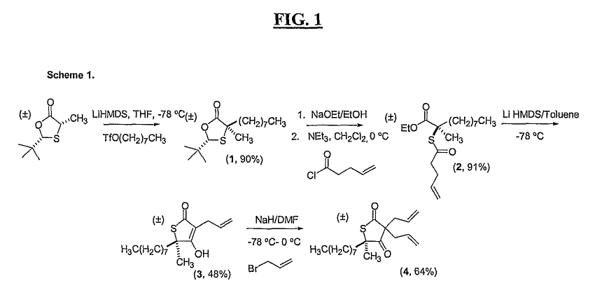

To 2-tert-butyl-5-methyl-5-octyl-[1,3]-oxathiolan-4-one (1, shown in FIG. 1, 2.2 g, 7.68 mmol) in EtOH (29 mL) was added NaOEt (2.1 M, 4.75 mL, 9.9 mmol) and the solution was allowed to stir at rt. After 40 min, the solution was poured into HCl (1 N, 30 mL) and extracted with Et₂O (3×30 mL). The combined organics were then washed with H₂O (5×30 mL), dried (MgSO₄), filtered and evaporated to give crude free thiol which was dissolved in CH₂Cl₂ (86 mL) and cooled to 0° C. NEt₃ (1.6 mL, 11.5 mmol) and 4-pentenoyl chloride (1.10 mL, 9.98 mmol) were added and the solution was allowed to stir at 0° C. for 1 h. NH₄Cl (sat. 150 mL) was added and the solution was extracted with CH₂Cl₂. The organic layer was dried (MgSO₄), filtered and evaporated. Flash chromatography 5% EtOAc/Hexanes gave 2-(4-pentenoyl)-sulfanyl-2-methyl-decanoic acid ethyl ester (2) (2.29 g, 91%). ¹H NMR (300 MHz, CDCl₃) δ 0.86 (t, J=6.9 Hz, 3H), 1.23 (m, 15H), 1.60 (s, 3H), 1.76-1.78 (m, 2H), 2.34-2.36 (m, 2H), 2.53-2.59 (m, 2H), 4.16 (q, J=7.2 Hz, 2H), 4.98 (d, J=10.3 Hz, 1H), 5.01 (d, J=17.6 Hz, 1H), 5.77 (ddt, J=10.3, 17.6, 6.3 Hz, 1H).

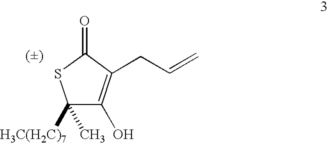

To 2-(4-pentenoyl)-sulfanyl-2-methyl-decanoic acid ethyl ester (2, 1.98 g, 6.04 mmol) in THF (91 mL) cooled to −78° C. was added LiHMDS (7.5 mL, 7.5 mmol) and the solution was allowed to slowly warm to −5° C. (2 h). The solution was then poured into HCl (1 N, 40 mL) and extracted with EtOAc (3×30 mL). The combined organics were dried (MgSO₄), filtered and evaporated. Flash chromatography (20% EtOAc/2% AcOH/Hexanes) gave pure 3-allyl-4-hydroxy-5-methyl-5-octyl-5-H-thiophe-2-one (3, 82 mg, 48%). ¹H NMR (300 MHz, CDCl₃) δ 0.85 (t, J=6.9 Hz, 3H), 1.24 (m, 12H), 1.65 (s, 3H), 1.81-1.86 (m, 2H), 3.02 (d, J=6.4 Hz, 2H), 5.12 (dq, J=10.6, 1.5 Hz, 1H), 5.20 (dq, J=17.3, 1.5 Hz, 1H), 5.84 (ddt, J=10.6, 17.3, 6.4 Hz, 1H). ¹³C NMR (100 MHz, CDCl₃) δ 14.1, 22.6, 25.2, 26.1, 26.9, 29.1, 29.3, 29.5, 31.8, 38.5, 57.5, 111.5, 117.4, 134.4, 180.8, 195.4.

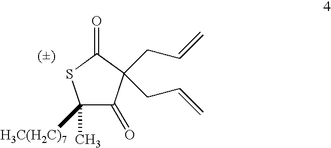

3,3-Diallyl-5-methyl-5-octyl-thiophene-2,4-dione (4). To 3-allyl-4-hydroxy-5-methyl-5-octyl-5-H-thiophe-2-one (3, 695 mg, 2.5 mmol) in DMF (14 mL) cooled to −40° C. was added NaH (60% in oil, 118 mg, 2.95 mmol) and the solution was allowed to warm to 0° C. and stir for 25 min. Allyl bromide (0.34 mL, 3.94 mmol) was added and the ice bath was removed allowing the reaction to warm to room temperature and stir 20 h. HCl (1 N, 30 mL) was added and the solution was extracted with Et₂O (3×30 mL). The combined organics were dried (MgSO₄), filtered and evaporated. Flash chromatographyl 2% EtOAc/Hex-110% EtOAc/Hex gave pure 4 (441 mg, 56%) and O-alkylated by-product (64 mg, 8%); overall yield (64%). C-alkylated product ¹H NMR (300 MHz, CDCl₃) δ 0.86 (t, J=6.5 Hz, 3H), 1.25 (m, 11H), 1.43-1.47 (m, 1H), 1.54 (s, 3H), 1.79-1.84 (m, 2H), 2.43-2.47 (m, 4H), 5.05-5.11 (m, 4H), 5.57-5.69 (2H). ¹³C NMR (100 MHz, CDCl₃) δ 14.1, 22.6, 25.1, 25.8, 29.1, 29.2, 29.5, 31.8, 40.2, 40.7, 41.3, 62.8, 64.8, 120.3, 120.4, 131.2, 131.2, 203.9, 213.5.

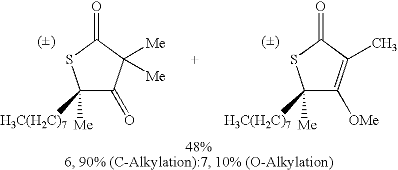

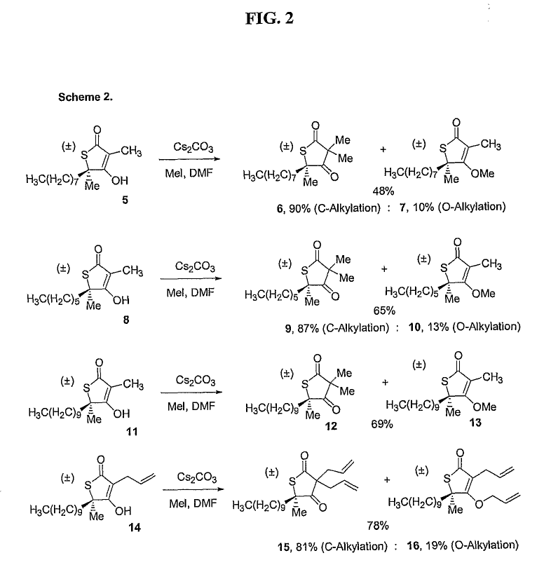

3,3,5-Trimethyl-5-octyl-thiophene-2,4-dione (6). To 5 (shown in FIG. 2 below and whose synthesis is described in PCT Application No. PCT US03/021700, 200 mg, 0.78 mmol) dissolved in DMF (4.3 mL) was added Cs₂CO₃ (304 mg, 0.94 mmol) and MeI (78 uL, 1.25 mmol). The solution was allowed to stir at rt for 1 h. The mixture was then poured into NH₄Cl/1N HCl (3:1, 20 mL) and extracted with Et₂O (3×15 mL). The Et₂O layer was then washed with H₂O (3×15 mL), dried (MgSO₄) filtered and evaporated to give crude 6/7. Flash chromatography 5% EtOAc/Hexanes to 20% EtOAc/Hexanes gave 6 (120 mg) and 7 (14 mg) 48% overall yield.

6: ¹H NMR (300 MHz, CDCl₃) δ 0.86 (t, J=6.99 Hz, 3H), 1.25 (m, 14H), 1.29 (s, 3H), 1.41-1.49 (m, 1H), 1.65 (s, 3H), 1.76-1.82 (m, 1H), 1.96-2.01 (m, 1H); ¹³C NMR (100 MHz, CDCl₃) δ 14.0, 22.2, 22.5, 24.4, 25.6, 28.1, 29.1, 29.2, 29.4, 31.7, 40.6, 53.6, 65.1, 204.9, 215.4.

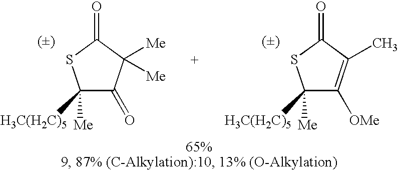

3,3,5-Trimethyl-5-hexyl-thiophene-2,4-dione (9). To 8 (shown in FIG. 2 below, 140 mg, 0.61 mmol) and MeI (65 uL, 1.06 mmol) following the above procedure but allowing the reaction to stir overnight at rt, was obtained 9 (83 mg) and 10 (13 mg) 65% overall yield after flash chromatography (2% EtOAc-5% EtOAc/Hexanes).

9: ¹H NMR (400 MHz, CDCl₃) δ 0.80 (t, J=6.8 Hz, 3H), 1.19 (m, 10H), 1.25 (s, 3H), 1.41-1.46 (m, 1H), 1.65 (s, 3H), 1.72-1.76 (m, 1H), 1.88-1.95 (m, 1H). ¹³C NMR (100 MHz, CDCl₃) δ 13.9, 22.2, 22.4, 24.4, 25.6, 28.1, 29.1, 31.4, 40.6, 53.6, 65.1, 204.9, 215.4.

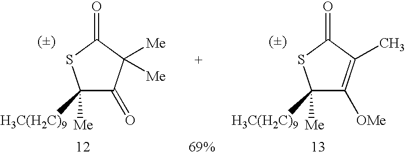

3,3,5-Trimethyl-5-decyl-thiophene-2,4-dione (12). To 11 (shown in FIG. 2 below, 209 mg, 0.74 mmol) and MeI (73 uL, 1.18 mmol) following the above procedure overnight was obtained 12 (151 mg, 69%) after flash chromatography 5% EtOAc/Hexanes. (O-alkylation was not recovered here but was present). ¹H NMR (400 MHz, CDCl₃) δ 0.83 (t, J=5.1 Hz, 3H), 1.21 (m, 18H), 1.26 (s, 3H), 1.42-1.46 (m, 1H), 1.70 (s, 3H), 1.71-1.74 (m, 1H), 1.89-1.96 (m, 1H). ¹³C NMR (100 MHz, CDCl₃) δ 14.0, 22.2, 22.6, 24.4, 25.6, 28.1, 29.2, 29.2, 29.4, 29.4, 29.4, 31.8, 40.6, 53.5, 65.0, 204.8, 215.4. ±

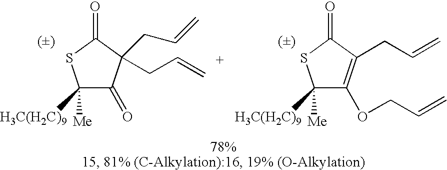

3,3-Diallyl-5-methyl-5-decyl-thiophene-2,4-dione (15). To 14 (shown in FIG. 2 below, 177 mg, 0.57 mmol) and allyl bromide (66 uL, 0.76 mmol) following the above procedure overnight was obtained 15 (126 mg) and 16 (30 mg) 78% overall after flash chromatography 5% EtOAc/Hexanes. ¹H NMR (300 MHz, CDCl₃) δ 0.85 (t, J=7.02 Hz, 3H), 1.23 (m, 15H), 1.40-1.50 (m, 1H), 1.53 (s, 3H), 1.75-1.86 (m, 2H), 2.37-2.50 (m, 4H), 5.03-5.09 (m, 4H), 5.52-5.66 (m, 2H).

### Biological and Biochemical Methods

Purification of FAS from ZR-75-1 Human Breast Cancer Cells.

Human FAS was purified from cultured ZR-75-1 human breast cancer cells obtained from the American Type Culture Collection. The procedure, adapted from Linn et al., 1981, and Kuhajda et al., 1994, utilizes hypotonic lysis, successive polyethyleneglycol (PEG) precipitations, and anion exchange chromatography. ZR-75-1 cells are cultured at 37° C. with 5% CO₂ in RPMI culture medium with 10% fetal bovine serum, penicillin and streptomycin.

Ten T150 flasks of confluent cells are lysed with 1.5 ml lysis buffer (20 mM Tris-HCl, pH 7.5, 1 mM EDTA, 0.1 mM phenylmethanesulfonyl fluoride (PMSF), 0.1% Igepal CA-630) and dounce homogenized on ice for 20 strokes. The lysate is centrifuged in JA-20 rotor (Beckman) at 20,000 rpm for 30 minutes at 4° C. and the supernatant is brought to 42 ml with lysis buffer. A solution of 50% PEG 8000 in lysis buffer is added slowly to the supernatant to a final concentration of 7.5%. After rocking for 60 minutes at 4° C., the solution is centrifuged in JA-20 rotor (Beckman) at 15,000 rpm for 30 minutes at 4° C. Solid PEG 8000 is then added to the supernatant to a final concentration of 15%. After the rocking and centrifugation is repeated as above, the pellet is resuspended overnight at 4° C. in 10 ml of Buffer A (20 mM K₂HPO₄, pH 7.4). After 0.45 μM filtration, the protein solution is applied to a Mono Q 5/5 anion exchange column (Pharmacia). The column is washed for 15 minutes with buffer A at 1 ml/minute, and bound material is eluted with a linear 60-ml gradient over 60 minutes to 1 M KCl. FAS (MW˜270 kD) typically elutes at 0.25 M KCl in three 0.5 ml fractions identified using 4-15% SDS-PAGE with Coomassie G250 stain (Bio-Rad). FAS protein concentration is determined using the Coomassie Plus Protein Assay Reagent (Pierce) according to manufacturer's specifications using BSA as a standard. This procedure results in substantially pure preparations of FAS (>95%) as judged by Coomassie-stained gels.

### Measurement of FAS Enzymatic Activity and Determination of the IC₅₀ of the Compounds

FAS activity is measured by monitoring the malonyl-CoA dependent oxidation of NADPH spectrophotometrically at OD₃₄₀ in 96-well plates (Dils et al and Arslanian et al, 1975). Each well contains 2 μg purified FAS, 100 mM K₂HPO₄, pH 6.5, 1 mM dithiothreitol (Sigma), and 187.5 μM β-NADPH (Sigma). Stock solutions of inhibitors are prepared in DMSO at 2, 1, and 0.5 mg/ml resulting in final concentrations of 20, 10, and 5 μg/ml when 1 μl of stock is added per well. For each experiment, cerulenin (Sigma) is run as a positive control along with DMSO controls, inhibitors, and blanks (no FAS enzyme) all in duplicate.

The assay is performed on a Molecular Devices SpectraMax Plus Spectrophotometer. The plate containing FAS, buffers, inhibitors, and controls are placed in the spectrophotometer heated to 37° C. Using the kinetic protocol, the wells are blanked on duplicate wells containing 100 μl of 100 mM K₂HPO₄, pH 6.5 and the plate is read at OD₃₄₀ at 10 sec intervals for 5 minutes to measure any malonyl-CoA independent oxidation of NADPH. The plate is removed from the spectrophotometer and malonyl-CoA (67.4 μM, final concentration per well) and alkynyl-CoA (61.8 μM, final concentration per well) are added to each well except to the blanks. The plate is read again as above with the kinetic protocol to measure the malonyl-CoA dependent NADPH oxidation. The difference between the Δ OD₃₄₀ for the malonyl-CoA dependent and non-malonyl-CoA dependent NADPH oxidation is the specific FAS activity. Because of the purity of the FAS preparation, non-malonyl-CoA dependent NADPH oxidation is negligible.

The IC₅₀ for the compounds against FAS is determined by plotting the Δ OD₃₄₀ for each inhibitor concentration tested, performing linear regression and computing the best-fit line, r² values, and 95% confidence intervals. The concentration of compound yielding 50% inhibition of FAS is the IC₅₀. Graphs of Δ OD₃₄₀ versus time are plotted by the SOFTmax PRO software (Molecular Devices) for each compound concentration. Computation of linear regression, best-fit line, r², and 95% confidence intervals are calculated using Prism Version 3.0 (Graph Pad Software).

### Crystal Violet Cell Growth Assay

The crystal violet assay measure cell growth but not cytotoxicity. This assay employs crystal violet staining of fixed cells in 96-well plates with subsequent solubilization and measurement of OD₄₉₀ on a spectrophotometer. The OD₄₉₀ corresponds to cell growth per unit time measured. Cells are treated with the compounds of interest or vehicle controls and IC₅₀ for each compound is computed.

To measure the cytotoxicity of specific compounds against cancer cells, 5×10⁴ MCF-7 human breast cancer cells, obtained from the American Type Culture Collection are plated per well in 24 well plates in DMEM medium with 10% fetal bovine serum, penicillin, and streptomycin. Following overnight culture at 37° C. and 5% CO₂, the compounds to be tested, dissolved in DMSO, are added to the wells in 1 μl volume at the following concentrations: 50, 40, 30, 20, and 10 μg/ml in triplicate. Additional concentrations are tested if required. 1 μl of DMSO is added to triplicate wells are the vehicle control. C75 is run at 10, and 5 μg/ml in triplicate as positive controls.

After 72 hours of incubation, cells are stained with 0.5 ml of Crystal Violet stain (0.5% in 25% methanol) in each well. After 10 minutes, wells are rinsed, air dried, and then solubilized with 0.5 ml 10% sodium dodecylsulfate with shaking for 2 hours. Following transfer of 100 μl from each well to a 96-well plate, plates are read at OD₄₉₀ on a Molecular Devices SpectraMax Plus Spectrophotometer Average OD₄₉₀ values are computed using SOFTmax Pro Software (Molecular Devices) and IC₅₀ values are determined by linear regression analysis using Prism version 3.02 (Graph Pad Software, San Diego).

### XTT Cytotoxicity Assay

The XTT assay is a non-radioactive alternative for the [⁵¹Cr] release cytotoxicity assay. XTT is a tetrazolium salt that is reduced to a formazan dye only by metabolically active, viable cells. The reduction of XTT is measured spectrophotometrically as OD₄₉₀-OD₆₅₀.

To measure the cytotoxicity of specific compounds against cancer cells, 9×10³ MCF-7 human breast cancer cells, obtained from the American Type Culture Collection are plated per well in 96 well plates in DMEM medium with 10% fetal bovine serum, insulin, penicillin, and streptomycin. Following overnight culture at 37° C. and 5% CO₂, the compounds to be tested, dissolved in DMSO, are added to the wells in 1 μl volume at the following concentrations: 80, 40, 20, 10, 5, 2.5, 1.25, and 0.625 μg/ml in triplicate. Additional concentrations are tested if required. 1 μl of DMSO is added to triplicate wells are the vehicle control. C75 is run at 40, 20, 10, 15, 12.5, 10, and 5 μg/ml in triplicate as positive controls.

After 72 hours of incubation, cells are incubated for 4 hours with the XTT reagent as per manufacturer's instructions (Cell Proliferation Kit II (XTT) Roche). Plates are read at OD₄₉₀ and OD₆₅₀ on a Molecular Devices SpectraMax Plus Spectrophotometer. Three wells containing the XTT reagent without cells serve as the plate blank. XTT data are reported as OD₄₉₀-OD₆₅₀. Averages and standard error of the mean are computed using SOFTmax Pro software (Molecular Dynamics).

The IC₅₀ for the compounds is defined as the concentration of drug leading to a 50% reduction in OD₄₉₀-OD₆₅₀ compared to controls. The OD₄₉₀-OD₆₅₀ are computed by the SOFTmax PRO software (Molecular Devices) for each compound concentration. IC₅₀ is calculated by linear regression, plotting the FAS activity as percent of control versus drug concentrations. Linear regression, best-fit line, r², and 95% confidence intervals are determined using Prism Version 3.0 (Graph Pad Software).

Measurement of [¹⁴C]acetate Incorporation into Total Lipids and Determination of IC₅₀ of Compounds

This assay measures the incorporation of [¹⁴C]acetate into total lipids and is a measure of fatty acid synthesis pathway activity in vitro. It is utilized to measure inhibition of fatty acid synthesis in vitro.

MCF-7 human breast cancer cells cultured as above, are plated at 5×10⁴ cells per well in 24-well plates. Following overnight incubation, the compounds to be tested, solubilized in DMSO, are added at 5, 10, and 20 μg/ml in triplicate, with lower concentrations tested if necessary. DMSO is added to triplicate wells for a vehicle control. C75 is run at 5 and 10 μg/ml in triplicate as positive controls. After 4 hours of incubation, 0.25 μCi of [¹⁴C]acetate (10 μl volume) is added to each well.

After 2 hours of additional incubation, medium is aspirated from the wells and 800 μl of chloroform:methanol (2:1) and 700 μl of 4 mM MgCl₂ is added to each well. Contents of each well are transferred to 1.5 Eppendorf tubes, and spun at full-speed for 2 minutes in a high-speed Eppendorf Microcentrifuge 5415D. After removal of the aqueous (upper) layer, an additional 700 μl of chloroform:methanol (2:1) and 500 μl of 4 mM MgCl₂ are added to each tube and then centrifuged for 1 minutes as above. The aqueous layer is removed with a Pasteur pipette and discarded. An additional 400 μl of chloroform:methanol (2:1) and 200 μl of 4 mM MgCl₂ are added to each tube, then centrifuged and aqueous layer is discarded. The lower (organic) phase is transferred into a scintillation vial and dried at 40° C. under N₂ gas. Once dried, 3 ml of scintillant (APB #NBC5104) is added and vials are counted for ¹⁴C. The Beckman Scintillation counter calculates the average cpm values for triplicates.

The IC₅₀ for the compounds is defined as the concentration of drug leading to a 50% reduction in [¹⁴C]acetate incorporation into lipids compared to controls. This is determined by plotting the average cpm for each inhibitor concentration tested, performing linear regression and computing the best-fit line, r² values, and 95% confidence intervals. The average cpm values are computed by the Beckman scintillation counter (Model LS6500) for each compound concentration. Computation of linear regression, best-fit line, r², and 95% confidence intervals are calculated using Prism Version 3.0 (Graph Pad Software).

### Carnitine Palmitoyltransferase-1 (CPT-1) Assay

CPT-1 catalyzes the ATP dependent transfer of long-chain fatty acids from acyl-CoA to acyl-carnitine that is inhibited by malonyl-CoA. As CPT-1 requires the mitochondrial membrane for activity, enzyme activity is measured in permeabilized cells or mitochondria. This assay uses permeabilized cells to measure the transfer of [methyl-¹⁴C]L-carnitine to the organically soluble acyl-carnitine deriviative.

MCF-7 cells are plated in DMEM with 10% fetal bovine serum at 10⁶ cells in 24-well plates in triplicate for controls, drugs, and malonyl-CoA. Two hours before commencing the assay, drugs are added at the indicated concentrations made from stock solutions at 10 mg/ml in DMSO, vehicle controls consist of DMSO without drug. Since malonyl-CoA cannot enter intact cells, it is only added in the assay buffer to cells that have not been preincubated with drugs. Following overnight incubation at 37° C., the medium is removed and replaced with 700 μl of assay buffer consisting of: 50 mM imidazole, 70 mM KCl, 80 mM sucrose, 1 mM EGTA, 2 mM MgCl₂, 1 mM DTT, 1 mM KCN, 1 mM ATP, 0.1% fatty acid free bovine serum albumin, 70 μM palmitoyl-CoA, 0.25 μCi [methyl-¹⁴C]L-carnitine, 40 μg digitonin with drug, DMSO vehicle control, or 20 μM malonyl-CoA. The concentrations of drugs and DMSO in the assay buffer is the same as used in the 2 hr preincubation. After incubation for 6 minutes at 37° C., the reaction is stopped by the addition of 500 μl of ice-cold 4 M perchloric acid. Cells are then harvested and centrifuged at 13,000×g for 5 minutes. The pellet is washed with 500 μl ice cold 2 mM perchloric acid and centrifuged again. The resulting pellet is resuspended in 800 μl dH₂O and extracted with 150 μl of butanol. The butanol phase is counted by liquid scintillation and represents the acylcarnitine derivative.

### Weight Loss Screen for Novel FAS Inhibitors

Balb/C mice (Jackson Labs) are utilized for the initial weight loss screening. Animals are housed in temperature and 12 hour day/night cycle rooms and fed mouse chow and water ad lib. Three mice are utilized for each compound tested with vehicle controls in triplicate per experiment. For the experiments, mice are housed separately for each compound tested three mice to a cage. Compounds are diluted in DMSO at 10 mg/ml and mice are injected intraperitoneally with 60 mg/kg in approximately 100 μl of DMSO or with vehicle alone. Mice are observed and weighed daily; average weights and standard errors are computed with Excel (Microsoft). The experiment continues until treated animals reach their pretreatment weights.

Select compounds are tested in animals housed in metabolic cages. Dosing of animals are identical to the screening experiments with three animals to a single metabolic cage. Animal weights, water and food consumption, and urine and feces production are measured daily.

### Antimicrobial Properties

A broth microdilution assay is used to assess the antimicrobial activity of the compounds. Compounds are tested at twofold serial dilutions, and the concentration that inhibits visible growth (OD₆₀₀ at 10% of control) is defined as the MIC. Microorganisms tested include Staphylococcus aureus (ATCC #29213), Enterococcus faecalis (ATCC #29212), Pseudomonas aeruginosa (ATCC #27853), and Escherichia coli (ATCC #25922). The assay is performed in two growth media, Mueller Hinton Broth and Trypticase Soy Broth.

A blood (Tsoy/5% sheep blood) agar plate is inoculated from frozen stocks maintained in T soy broth containing 10% glycerol and incubated overnight at 37° C. Colonies are suspended in sterile broth so that the turbidity matches the turbidity of a 0.5 McFarland standard. The inoculum is diluted 1:10 in sterile broth (Mueller Hinton or Trypticase soy) and 195 ul is dispensed per well of a 96-well plate. The compounds to be tested, dissolved in DMSO, are added to the wells in 5 ul volume at the following concentrations: 25, 12.5, 6.25, 3.125, 1.56 and 0.78 ug/ml in duplicate. Additional concentrations are tested if required. 5 ul of DMSO added to duplicate wells are the vehicle control. Serial dilutions of positive control compounds, vancomycin (E. faecalis and S. aureus) and tobramycin (E. coli and P. aeruginosa), are included in each run.

After 24 hours of incubation at 37° C., plates are read at OD₆₀₀ on a Molecular Devices SpectraMax Plus Spectrophotometer. Average OD₆₀₀ values are computed using SOFTmax Pro Software (Molecular Devices) and MIC values are determined by linear regression analysis using Prism version 3.02 (Graph Pad Software, San Diego). The MIC is defined as the concentration of compound required to produce an OD₆₀₀ reading equivalent to 10% of the vehicle control reading.

### β-Oxidation Assay—Isolation of Acid Soluble Products

A 24 well plate with 1 ml per well was prepared with 2.5×105 cells/well. The cells were incubated O/N.

The next day, a solubilized palmitate solution was prepared. 50 μls of (1-¹⁴C) Palmitic acid was added to a 2 ml centrifuge tube and dried under nitrogen gas. 2 mls of α-CD (α-Cyclodextran)-10 mg/ml in 10 mM Tris were added. This solution was incubated in a 37° C. water bath for 30 minutes.

A hot mix was prepared by adding 25 μls of this solution to 2.5 μls of 200 μM Carnitine and 222.5 μls of serum free medium that is used for cells.

The cells were then treated with the test compound in triplicate, and incubated at 37° C. for 60 minutes. The medium was removed and 250 μls of the hot mix were added. The test compound was added again, and further incubated at 37° C. for 60 minutes. The reaction was stopped with 50 μls of 2.6 N HClO₄. The contents of the plate were transferred to a 1.5 ml centrifuge tube, and 50 μls of 4N KOH were then added, and the tube incubated in a 60° C. water bath for 30 min. Sodium acetate (1M, 75 μls) and sulfuric acid (3N, 50 μls) were added to the solution and vortexed. The tube was spun for 7 minutes at 1000 rpm at room temperature. A portion (225 μls) was removed, and the following were added (vortex after each addition, twice at the end): 938 μls of 1:1 Chloroform:Methanol; 468 μls of Chloroform; 281 μls distilled Water. The tubes were spun at 1000 rpm for 5 minutes. The upper phase was removed into a large glass scintillation vial and 5 mls of Budget solvent (scintillation liquid) was added. The tubes were well vortexed. Finally, C14 was counted for one minute.

### Results of the Biological Testing

| | |
|---|---|
| | 4 |
| 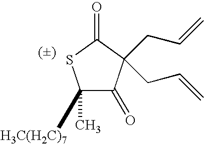 | |

| | | | | Cr. Violet |
|---|---|---|---|---|
| FAS (IC₅₀) | ¹⁴C (IC₅₀) | XTT (IC₅₀) | | (IC₅₀) |
| Neg | Neg | 40.5 ± 14.8 | ug/ml (M) | 15.0 ± 7.7 |

| | | |
|---|---|---|
| 285 ug/ml (SB) | 53.7 ± 1.0 | ug/ml (O) |

| CPT I Stim | Weight Loss | | |
|---|---|---|---|
| 125% of control | 60 mg/kg: 7.9% and 8.0% and 6.8% (day 1) | | |
| at 20 ug/ml | | | |
| SA/MH (MIC) | SA/Tsoy (M | PSAE/MH (MIC) | PSAE/Tsoy |
| Neg | 98 ug/ml | Neg | Neg |
| EF/MH (MIC) | EF/Tsoy (MI | Ecoli/MH (MIC) | Ecoli/Tsoy |
| Neg | 169 ug/ml | Neg | Neg |

Beta Oxidation

| | 1.25 | 2.5 | 5 | 10 | 20 | 40 |
|---|---|---|---|---|---|---|
| | ug/ml | ug/ml | ug/ml | ug/ml | ug/ml | ug/ml |
| Compound 4 | 94 | 114 | 120 | 163 | | |
| Compound 4 | | | 154 | 177 | 147 | 101 |
| Compound 4 | 151 | 163 | 177 | 184 | | |

| | |
|---|---|
| | 6 |
| 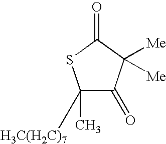 | |

| | | | | Cr. Violet |
|---|---|---|---|---|
| FAS (IC₅₀) | ¹⁴C (IC₅₀) | XTT (IC₅₀) | | (IC₅₀) |
| Neg (SB) | Not Tested | 23.0 | ug/ml (M) | Not Tested |

| | | |
|---|---|---|
| Sol | >80 | ug/ml (O) |

| CPT I Stim | Weight Loss |
|---|---|
| Not Tested | Not Tested |
| FAO SC 150 | FAO MAx |
| Neg | 141% at |
| | 6.25 ug/ml |

Beta Oxidation

| | 0.097 | 0.39 | 1.56 | 6.25 | 25 | 100 |
|---|---|---|---|---|---|---|
| | ug/ml | ug/ml | ug/ml | ug/ml | ug/ml | ug/ml |
| Compound 6 | 100 | 110 | 110 | 121 | 57 | 19 |
| Compound 6 | 86 | 93 | 110 | 141 | 52 | 29 |
| Compound 6 | | | 102 | 130 | 53 | |

| | |
|---|---|
| | 9 |
| 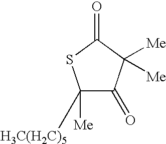 | |

| | | | | Cr. Violet |
|---|---|---|---|---|
| FAS (IC₅₀) | ¹⁴C (IC₅₀) | XTT (IC₅₀) | | (IC₅₀) |
| Neg (SB) | Not Tested | 5.3 | ug/ml (M) | Not Tested |

| | | |
|---|---|---|
| Limited by | 14.0 | ug/ml (O) |

| CPT I Stim | Weight Loss |
|---|---|
| Not Tested | Not Tested |
| FAO SC 150 | FAO MAx |
| Neg | 115% at 6.25 ug/ml |

Beta Oxidation

| | 0.097 | 0.39 | 1.56 | 6.25 | 25 | 100 |
|---|---|---|---|---|---|---|
| | ug/ml | ug/ml | ug/ml | ug/ml | ug/ml | ug/ml |
| Compound 9 | 96 | 99 | 97 | 115 | 58 | 27 |

| | |
|---|---|
| | 15 |
| 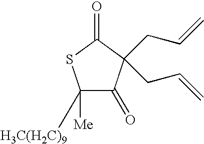 | |

| | | | | | Cr. Violet |
|---|---|---|---|---|---|
| FAS (IC₅₀) | | ¹⁴C (IC₅₀) | XTT (IC₅₀) | | (IC₅₀) |
| 282 | ug/ml (SB) | Not Tested | 71.8 | ug/ml (M) | Not Tested |
| | | | >80 | ug/ml (O) | |

| CPT I Stim | | Weight Loss |
|---|---|---|
| Not Tested | | 60 mg/kg: 3.5% (day 2) |
| FAO SC 150 | | FAO MAx |
| 16.0 | ug/ml | 165% at 25 |

Beta Oxidation

| | 0.097 | 0.39 | 1.56 | 6.25 | 25 | 100 |
|---|---|---|---|---|---|---|
| | ug/ml | ug/ml | ug/ml | ug/ml | ug/ml | ug/ml |
| Compound 15 | 105 | 114 | 127 | 138 | 165 | 150 |

| | |
|---|---|
| | 12 |
| 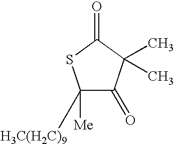 | |

| | | | | | Cr. Violet |
|---|---|---|---|---|---|
| FAS (IC₅₀) | | ¹⁴C (IC₅₀) | XTT (IC₅₀) | | (IC₅₀) |
| 255 | ug/ml (SB) | Not Tested | 23.2 | ug/ml (M) | Not Tested |
| | | | >80 | ug/ml (O) | |

| CPT I Stim | | Weight Loss |
|---|---|---|
| Not Tested | | 60 mg/kg: 9.0% (day 2) |
| FAO SC 150 | | FAO MAx |
| 1.4 | ug/ml | 154 at 1.56 |

Beta Oxidation

| | 0.097 | 0.39 | 1.56 | 6.25 | 25 | 100 |
|---|---|---|---|---|---|---|
| | ug/ml | ug/ml | ug/ml | ug/ml | ug/ml | ug/ml |
| Compound 12 | 100 | 120 | 154 | 141 | 75 | 68 |

We claim:

1. A compound of formula: 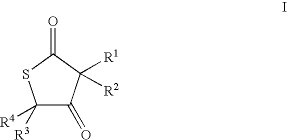
wherein:
R¹ and R², the same or different from each other, are H, C₁-C₂₀ alkyl, cycloalkyl, alkenyl, aryl, arylalkyl, or alkylaryl, CH₂COR⁵, —CH₂C(O)NR⁵, —C(O)R⁵, or —CH₂OR⁵, and can optionally contain halogen atoms, where R⁵ is a C₁-C₁₂ alkyl group.
R³ and R⁴, the same or different from each other, are H, C₁-C₂₀ alkyl, cycloalkyl, alkenyl, aryl, arylalkyl, or alkylaryl;
with the proviso that when R⁴—(CH₂)₇CH₃, R³ is methyl, and R¹ is —CH₃, R² is not —CH₂—CH═CH₂,
and with the further proviso that when R⁴—CH₃, R³ is H, R¹ is —CH₃, R² is not —CH₃, or —CH═C(CH₃)CH₂CH₂CH═C(CH₃)₂.

2. A compound according to claim 1, wherein R¹ and R² are each independently C₁-C₁₂ alkyl.

3. A compound according to claim 2, wherein R¹ and R² are each —CH₂—CH═CH₂.

4. A compound according to claim 1, wherein R³ and R⁴ are each independently a C₁-C₁₂ alkyl group.

5. A compound according to claim 4, wherein R⁴ is a C₁-C₆ alkyl group.

6. A compound according to claim 4, wherein R⁴ is —CH₃.

7. A compound according to claim 1, wherein the compound has the structure:

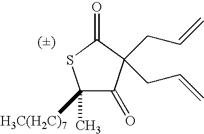

8. A compound according to claim 1, wherein the compound has the structure:

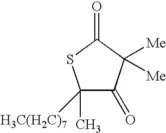

9. A compound according to claim 1, wherein the compound has the structure:

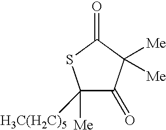

10. A compound according to claim 1, wherein the compound has the structure:

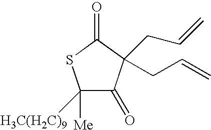

11. A compound according to claim 1, wherein the compound has the structure:

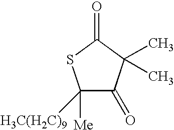

12. A pharmaceutical composition comprising a pharmaceutical diluent and a compound of formula II: 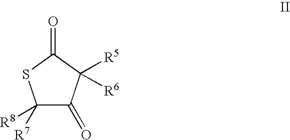
wherein:
R⁵ and R⁶, the same or different from each other, are H, C₁-C₂₀ alkyl, cycloalkyl, alkenyl, aryl, arylalkyl, or alkylaryl, —CH₂COR⁹, —CH₂C(O)NR⁹, —C(O)R⁹, or —CH₂OR⁹, and can optionally contain halogen atoms, where R⁹ is a C₁-C₁₂ alkyl group;
R⁷ and R⁸, the same or different from each other, are H, C₁-C₂₀ alkyl, cycloalkyl, alkenyl, aryl, arylalkyl, or alkylaryl.

13. A pharmaceutical composition according to claim 12, comprising a compound of formula I: 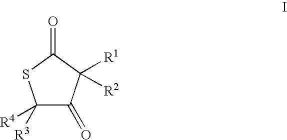
wherein:
R¹ and R², the same or different from each other, are H, C₁-C₂₀ alkyl, cycloalkyl, alkenyl, aryl, arylalkyl, or alkylaryl, —CH₂COR⁵, —CH₂C(O)NR⁵, —C(O)R⁵, or —CH₂OR⁵, and can optionally contain halogen atoms, where R⁵ is a C₁-C₁₂ alkyl group.
R³ and R⁴, the same or different from each other, are H, C₁-C₂₀ alkyl, cycloalkyl, alkenyl, aryl, arylalkyl, or alkylaryl;
with the proviso that when R⁴═—(CH₂)₇CH₃, R³ is methyl, R¹ is —CH₃, R² is not —CH₂—CH═CH₂,
and with the further proviso that when R⁴═—CH₃, R³ is H, R¹ is —CH₃, R² is not —CH₃, or —CH═C(CH₃)CH₂CH₂CH═C(CH₃)₂.
comprising compounds of formula I and a pharmaceutical diluent.

14. A method of inducing weight loss in animals and humans by administering a pharmaceutical composition of claim 12.

15. A method of stimulating the activity of CPT-1 by administering to humans or animals a pharmaceutical composition of claim 12.

16. A method of inhibiting the synthesis of neuropeptide Y in humans or animals by administering a pharmaceutical composition of claim 12.

17. A method of inhibiting fatty acid synthase activity in humans or animals by administering a pharmaceutical composition of claim 12.

18. A method of treating cancer in animals and humans by administering a pharmaceutical composition of claim 12.

19. A method of preventing the growth of cancer cells in animals and humans by administering a pharmaceutical composition of claim 12.

20. A method of inhibiting growth of invasive microbial cells by administering a pharmaceutical composition of claim 12.
